SEQUENCE LISTING (21 sequences)

SEQ ID NO: 1 - DNA, 73 bp, synthetic construct
  misc_feature 1..73: note = Synthetic Construct
  source 1..73: mol_type = other DNA
ggccgcgtggcctaatggataaggcgtctgacttcagatcagaagattgcaggttcgagtcctgccgcggtcg

SEQ ID NO: 2 - DNA, 73 bp, synthetic construct
  misc_feature 1..73: note = Synthetic Construct
  source 1..73: mol_type = other DNA
gaccgcgtggcctaatggataaggcgtctgacttcagatcagaagattgagggttcgagtcccttcgtggtcg

SEQ ID NO: 3 - DNA, 73 bp, synthetic construct
  misc_feature 1..73: note = Synthetic Construct
  source 1..73: mol_type = other DNA
ggccgtgtggcctaatggataaggcgtctgacttcagatcaaaagattgcaggtttgagttctgccacggtcg

SEQ ID NO: 4 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 5 - DNA, 473 bp, synthetic construct
  misc_feature 1..473: note = Synthetic Construct
  source 1..473: mol_type = other DNA
gcgcgaccaataggagggccgccaatggagcggtccgcccgcggggggagaagggggacttctgggtgggctctccctgcggaacgcgcgaaccaaaggccaacctccccttctcaaggagcaggtggattggtcccgagctagctggtgggcggaggtgacgtttttataagttgctcaagagacggtaacaaccgacgggccgcgtggcctaatggataaggcgtctgacttcagatcagaagattgcaggttcgagtcctgccgcggtcgaagggaggttatgattaacttttagtttattcctccctcaggaacgaagtattgggacaatgtgaacgtagtcgccgccgattcccaccgcacttcaaagatgtgggaacgccaagatccgcggaagtaaccacgcccagcaagtccctgcgagattgcccgcctacgtgtctcagcggaggcacatttctaaaatgtac

SEQ ID NO: 6 - DNA, 473 bp, synthetic construct
  misc_feature 1..473: note = Synthetic Construct
  source 1..473: mol_type = other DNA
cgcggtcgcaattcccagggaggctcagagactagtccaggtgccgctccgctcgggggtgactaggcgggcggctggggccagaggaagggtgagcgcttcgctcaacaggcggccagggtgcgagcagtgaagctgcggcacgccggagcgtttaatggccatcaaattggcctctctaggaggtagctgcagccggagaccgcgtggcctaatggataaggcgtctgacttcagatcagaagattgagggttcgagtcccttcgtggtcggaacgttttaatccctgcaactataatcttccctcccttgttttagacccagaaacgcctcccaatccctccacaccgcggctcccgggatccgaagctggggagctgcttctgtccaatcaggctttgcatcccgggacgcttccggttatggcgtcattatgcagccagggtaagtagggccccatttcttgttgtct

SEQ ID NO: 7 - DNA, 473 bp, synthetic construct
  misc_feature 1..473: note = Synthetic Construct
  source 1..473: mol_type = other DNA
aggcagccacatggggagtccacactgggaagtggaagcctggggaaccctaggtaaaggtggcctgcctgatgcaggatttacaatgtgagctggtttttctctatcttaggaagagaagaggaaaactttctgctaatgttatcggtggaggccatccaggctcttggcattttgaacaaagacttggccaaccagctggccgtgtggcctaatggataaggcgtctgacttcagatcaaaagattgcaggtttgagttctgccacggtcgtggaggggttatattttgttgggtgcggtggcttatgcctgtaatcccagcatcttgggaggccgaggaaagcggatcacctgaggtcgggagtttgaggccagcctggccaacatggagaaaccccatctctactaaaaatacaaaattagccgggcgtggtggcgcatgcctgtaatcccagctactcggtaggctca

SEQ ID NO: 8 - DNA, 165 bp, synthetic construct
  misc_feature 1..165: note = Synthetic Construct
  source 1..165: mol_type = other DNA
gaggtgacgtttttataagttgctcaagagacggtaacaaccgacgggccgcgtggcctaatggataaggcgtctgacttcagatcagaagattgcaggttcgagtcctgccgcggtcgaagggaggttatgattaacttttagtttattcctccctcaggaacg

SEQ ID NO: 9 - DNA, 165 bp, synthetic construct
  misc_feature 1..165: note = Synthetic Construct
  source 1..165: mol_type = other DNA
ttaatggccatcaaattggcctctctaggaggtagctgcagccggagaccgcgtggcctaatggataaggcgtctgacttcagatcagaagattgagggttcgagtcccttcgtggtcggaacgttttaatccctgcaactataatcttccctcccttgttttag

SEQ ID NO: 10 - DNA, 389 bp, synthetic construct
  misc_feature 1..389: note = Synthetic Construct
  source 1..389: mol_type = other DNA
cgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgggcggagggagacggtaacaaccgacgggccgcgtggcctaatggataaggcgtctgacttcagatcagaagattgcaggttcgagtcctgccgcggtcgaagggaggttatgattaacttttagtttattcctccctcaggaacg

SEQ ID NO: 11 - DNA, 389 bp, synthetic construct
  misc_feature 1..389: note = Synthetic Construct
  source 1..389: mol_type = other DNA
cgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgggcggaggggaggtagctgcagccggagaccgcgtggcctaatggataaggcgtctgacttcagatcagaagattgagggttcgagtcccttcgtggtcggaacgttttaatccctgcaactataatcttccctcccttgttttag

SEQ ID NO: 12 - DNA, 51 bp, synthetic construct
  misc_feature 1..51: note = Synthetic Construct
  source 1..51: mol_type = other DNA
acaagccttttcagctttagagggtgagcaaaggatgtgggatctgagaac

SEQ ID NO: 13 - DNA, 51 bp, synthetic construct
  misc_feature 1..51: note = Synthetic Construct
  source 1..51: mol_type = other DNA
ctgagacctctaagagccttatcttgatttgaagggatgagggtggttgtg

SEQ ID NO: 14 - protein, 1998 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 15 - protein, 1981 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 16 - DNA, 72 bp, synthetic construct
  misc_feature 1..72: note = Synthetic Construct
  source 1..72: mol_type = other DNA
ggtcccatggtgtaatggttagcactctggactttaaatccagcgatccgagttcaaatctcggtgggacct

SEQ ID NO: 17 - DNA, 72 bp, synthetic construct
  misc_feature 1..72: note = Synthetic Construct
  source 1..72: mol_type = other DNA
ggtcccatggtgtaatggttagcactctggactttaaatccagcaatccgagttcgaatctcggtgggacct

SEQ ID NO: 18 - DNA, 72 bp, synthetic construct
  misc_feature 1..72: note = Synthetic Construct
  source 1..72: mol_type = other DNA
ggccccatggtgtaatggttagcactctggactttaaatccagcgatccgagttcaaatctcggtgggacct

SEQ ID NO: 19 - DNA, 473 bp, synthetic construct
  misc_feature 1..473: note = Synthetic Construct
  source 1..473: mol_type = other DNA
cggaggagcgcaggaagacatgatatccccgggaccagaaacgccgggaacacgaaggacgcagaagccctcgccacgccgcccgtggggccccggtccagggcccagtccctcaaacacggacaccgctgccgctctggaaaggccgcccagagccctcctagcttgaggagactcgcggccgccttaagaagccagcaggtcccatggtgtaatggttagcactctggactttaaatccagcgatccgagttcaaatctcggtgggacctcacgagactttttggtcctacaattcctgttttggagaccgcttccggcagcagccacctcgggctgtgtgggtagcctcactgacctggcctacagcggcttggggtccctccccctctctcaagatgttttcatttcagggccgttctgggggtgtcagtgcgcacgcgcgagagtcgggcagaaaaataaatgtgttg

SEQ ID NO: 20 - DNA, 472 bp, synthetic construct
  misc_feature 1..472: note = Synthetic Construct
  source 1..472: mol_type = other DNA
caaaagcgcaggaaagagtgaagagatctccctgaattttattttgacagttactccttcttcagcagcaaagcgtcagctttcctgtcagcgtgagtctcactgaagcctgacctaactctttcggatcggaactagaaagtttattatcaaggaggtatttaaccacgactatacttacctactgtacccttgtggttggtcccatggtgtaatggttagcactctggactttaaatccagcaatccgagttcgaatctcggtgggacctttcaaaggtgaacgttttacagttcctggcttggcctctgaatgtgggaaaaattcgttcctccgcttcccatcgaattctcgtcgaaaacaactttgtggtcgcgtgcacggtattgaactctccccttctatccaagcatgaaagtaccaagctctttcgcctagaagaacgctttcaaagcgtatcaaattcacggg

SEQ ID NO: 21 - DNA, 472 bp, synthetic construct
  misc_feature 1..472: note = Synthetic Construct
  source 1..472: mol_type = other DNA
actcaaacgggttcaagttttttcagtttctaagacggacttggtccagataggtttgtagtgcatgtgagtctcttggagcttctgctacatgggatgcagatagtattaatttaataaatattaagagaatactgcatcgaatagacatgctctgtaatcagtatttttataaattcatgaaattggtggcggaaggcggccccatggtgtaatggttagcactctggactttaaatccagcgatccgagttcaaatctcggtgggacctgtcctgctttttgtgacccatccttcgttcgtctcttaaatcatcaacacacagcgatctgtggatttactttctcattcacggtttcttcttcggattagcaagtgtcatgtaactcctgactgtcatttctagcttctgagtagtatcatgtccttgtacattcttggtcttttctcttagcatccacacttccctga

(19) United States
(12) Patent Application Publication
(10) Pub. No.: US 2024/0050594 A1
(43) Pub. Date: Feb. 15, 2024

(54) METHODS AND COMPOSITIONS FOR TREATING A PREMATURE STOP CODON-MEDIATED DISORDER

(71) Applicants: CASE WESTERN RESERVE UNIVERSITY, Cleveland, OH (US); WHITEHEAD INSTITUTE FOR BIOMEDICAL RESEARCH, Cambridge, MA (US)

(72) Inventors: Jeffery M. Coller, Cleveland, OH (US); Thomas Sweet, Cleveland, OH (US); Harvey Lodish, Cambridge, MA (US)

(21) Appl. No.: 18/130,278

(22) Filed: Apr. 3, 2023

Related U.S. Application Data
(63) Continuation of application No. 17/151,126, filed on Jan. 16, 2021, now Pat. No. 11,617,802.
Application No. 17/151,126 is a continuation of application No. 16/665,526, filed on Oct. 28, 2019, now Pat. No. 10,905,778.
Application No. 16/665,526 is a continuation of application No. PCT/US2019/053260, filed on Sep. 26, 2019.
(60) Provisional application No. 62/805,793, filed on Feb. 14, 2019.
(60) Provisional application No. 62/747,646, filed on Oct. 18, 2018.
(60) Provisional application No. 62/736,834, filed on Sep. 26, 2018.

(51) Int. Cl. A61K 48/00; A61P 25/08

(52) U.S. Cl. CPC A61K 48/0066; A61P 25/08

(57) ABSTRACT

Modified tRNAs can be used to express in a mammalian cell a functional gene product encoded by a gene containing a premature stop codon and/or to treat a disease mediated by a premature stop codon.

### RELATED APPLICATION

This application claims priority from U.S. Provisional Application Nos. 62/736,834, filed Sep. 26, 2018, and 62/805,793, filed Feb. 14, 2019, the subject matter of which are incorporated herein by reference in their entirety.

### FIELD OF THE INVENTION

This disclosure relates generally to methods and compositions for expressing a gene product encoded by a gene containing a premature stop codon and/or treating a disorder mediated by a premature stop codon.

### BACKGROUND

Protein synthesis is directed by a genetic code that includes 61 three-base-pair codons encoding amino acids that are incorporated into the protein being synthesized and 3 three-base-pair codons (referred to as stop or termination codons) that terminate the synthesis of a protein. When a nucleic acid sequence encoding a protein is mutated to contain a premature stop codon rather than a codon for the next amino acid, the resulting protein is prematurely terminated, which is often nonfunctional or less functional than the untruncated or full length protein. Such mutations, termed nonsense mutations, are often associated with, or are a causative agent in numerous different genetic diseases.

A number of disorders are associated with, or are caused by nonsense mutations. These include β-thalassemia, Choroideremia (CHM), Cystic Fibrosis, Dravet Syndrome, Duchenne Muscular Dystrophy, Hurler Syndrome, KIF1A, a Lysosomal Storage Disease (e.g., Maroteaux-Lamy Syndrome, Niemann Pick Disease, and Sanfilippo Syndrome), Marfan Syndrome, Smith-Lemli-Opitz Syndrome, and Spinal Muscular Atrophy.

Dravet Syndrome is a rare and catastrophic form of intractable epilepsy that begins in infancy. Initially, patients experience prolonged seizures. In their second year, additional types of seizure begin to occur, which typically coincide with a developmental decline, possibly due to repeated cerebral hypoxia. This leads to poor development of language and motor skills. Mutations in SCN1A (encoding the voltage-gated sodium channel a subunit), SCN1B (encoding the voltage-gated sodium channel β1 subunit), SCN2A, SCN3A, SCN9A, GABRG2 (encoding the γ-aminobutyric acid receptor γ2 subunit), GABRD (encoding the γ-aminobutyric acid receptor Δ subunit) and/or PCDH19 genes have been linked to Dravet Syndrome.

For example, Dravet syndrome may be caused by a nonsense mutation in the gene resulting in a premature stop codon and a lack of or reduced amount of untruncated or functional protein. The SCN1A gene normally codes for the neuronal voltage-gated sodium channel a subunit, Na(V)1.1. In mouse models, loss-of-function mutations in SCN1A have been observed to result in a decrease in sodium currents and impaired excitability of GABAergic interneurons of the hippocampus.

Accordingly, there is a need in the art for improved compositions and methods for treating diseases mediated by premature stop codons, including Dravet syndrome.

### SUMMARY

One aspect of this disclosure provides a method of expressing in a mammalian cell a functional gene product encoded by a gene containing a premature stop codon, e.g., a SCN1A gene. The method includes introducing into the cell an effective amount of an expression vector capable of expressing a tRNA that (i) comprises an anticodon that hybridizes to the premature stop codon, and (ii) is capable of being aminoacylated with an amino acid, so that the tRNA, when expressed in the cell and aminoacylated with the amino acid, hybridizes to the premature stop codon and permits the amino acid to be incorporated into the gene product at a position that would otherwise result in a truncated gene product caused by the premature stop codon.

Another aspect of this disclosure provides a method of expressing in a mammalian cell a functional gene product encoded by a gene containing a premature stop codon, e.g., a SCN1A gene. The method includes introducing into the cell an effective amount of a tRNA that (i) comprises an anticodon that hybridizes to the premature stop codon, and (ii) is capable of being aminoacylated with an amino acid, so that the tRNA, when introduced into the cell and aminoacylated with the amino acid, hybridizes to the premature stop codon and permits the amino acid to be incorporated into the gene product at a position that would otherwise result in a truncated gene product caused by the premature stop codon.

In certain embodiments of any of the foregoing methods, the gene can be an α-L-iduronidase, ARSB, β-globin, CFTR, CHM, DHCR7, dystrophin, fibrin-1 (FBN1), KIF1A, NAGLU, SCN1A, SMN1, or SMPD1 gene. In certain embodiments, the gene is a SCN1A gene.

In another aspect, this disclosure provides a method of increasing in a cell voltage-gated sodium channel activity encoded by a SCN1A gene containing a premature stop codon. The method includes introducing into the cell an effective amount of an expression vector capable of expressing a tRNA that (i) comprises an anticodon that hybridizes to the premature stop codon, and (ii) is capable of being aminoacylated with an amino acid, so that the tRNA, when expressed in the cell and aminoacylated with the amino acid, hybridizes to the premature stop codon and permits the amino acid to be incorporated into the SCN1A gene product at a position that would otherwise result in a truncated SCN1A gene product caused by the premature stop codon.

In another aspect, this disclosure provides a method of increasing in a cell voltage-gated sodium channel activity encoded by a SCN1A gene containing a premature stop codon. The method includes introducing into the cell an effective amount of a tRNA that (i) comprises an anticodon that hybridizes to the premature stop codon, and (ii) is capable of being aminoacylated with an amino acid, so that the tRNA, when introduced into the cell and aminoacylated with the amino acid, hybridizes to the premature stop codon and permits the amino acid to be incorporated into the SCN1A gene product at a position that would otherwise result in a truncated SCN1A gene product caused by the premature stop codon.

In certain embodiments of any of the foregoing methods, the cell contains less truncated gene product than a cell without the tRNA. In certain embodiments, the cell contains a greater amount of functional gene product than a cell without the tRNA.

In certain embodiments of any of the foregoing methods, the gene is a SCN1A gene, and the SCN1A gene product produced with the tRNA is a functional SCN1A gene product. In certain embodiments, the functional SCN1A gene product has greater activity than the truncated SCN1A gene product. In certain embodiments, the functional SCN1A gene product is the Naᵥ1.1 protein. In certain embodiments, the functional SCN1A gene product comprises a polypeptide having an amino acid sequence of SEQ ID NO: 4, SEQ ID NO: 14, or SEQ ID NO: 15. In certain embodiments, the functional SCN1A gene product comprises a polypeptide having an amino acid sequence of SEQ ID NO: 4.

In certain embodiments of any of the foregoing methods, the cell is a human cell. In certain embodiments, the cell is a central nervous system cell, e.g., a neuron. In certain embodiments, the tRNA becomes aminoacylated in the cell.

In another aspect, this disclosure provides a method of treating a premature stop codon-mediated disorder, e.g., Dravet syndrome, in a subject in need thereof, wherein the subject has a gene with a premature stop codon, e.g., a SCN1A gene. The method includes administering to the subject an effective amount of an expression vector capable of expressing a tRNA that (i) comprises an anticodon that hybridizes to the premature stop codon, and (ii) is capable of being aminoacylated with an amino acid, thereby to treat the disorder in the subject.

In another aspect, this disclosure provides a method of treating a premature stop codon-mediated disorder, e.g., Dravet syndrome, in a subject in need thereof, wherein the subject has a gene with a premature stop codon, e.g., a SCN1A gene. The method includes administering to the subject an effective amount of a tRNA that (i) comprises an anticodon that hybridizes to the premature stop codon, and (ii) is capable of being aminoacylated with an amino acid, thereby to treat the disorder in the subject.

In certain embodiments of any of the foregoing methods of treatment, the premature stop codon-mediated disorder is selected from β-thalassemia, Choroideremia (CHM), Cystic Fibrosis, Dravet Syndrome, Duchenne Muscular Dystrophy, Hurler Syndrome, KIF1A, a Lysosomal Storage Disease (e.g., Maroteaux-Lamy Syndrome, Niemann Pick Disease, and Sanfilippo Syndrome), Marfan Syndrome, Smith-Lemli-Opitz Syndrome, and Spinal Muscular Atrophy. In certain embodiments, the premature stop codon-mediated disorder is Dravet syndrome. In certain embodiments, the premature stop codon-mediated disorder is Dravet syndrome and the gene is SCN1A.

In other embodiments of any of the foregoing methods of treatment, the premature stop codon-mediated disorder is selected from epilepsy disorders, epileptic encephalopathies, Dravet Syndrome, Lennox-Gastaut Syndrome, Kleefstra Syndrome, Duchenne Muscular Dystrophy; KCNQ2 Encephalopathy, SYNGAP1 Encephalopathy, Parkinson's with GBA, CDKL5, SLC6A1, BRMUTD, Sotos Syndrome, GLUT1 Deficiency Syndrome and any other premature stop codon-mediated disorder associated with a central nervous system (CNS)-related disorder. In some embodiments, the premature stop codon-mediated disorder is selected from epilepsy disorder or epileptic encephalopathies, including Dravet Syndrome and Lennox-Gastaut Syndrome.

In still other embodiments of any of the foregoing methods of treatment, the premature stop codon-mediated disorder is selected 5q-syndrome, Adams-Oliver syndrome 1, Alagille syndrome 1, Autoimmune lymphoproliferative syndrome type 1A, Carney complex type I, CHARGE syndrome, Coffin-Siris Syndrome, Duane Syndrome, Cystic Fibrosis, Marfan Syndrome, Ehlers-Danlos Syndrome, Feingold Syndrome 1, Denys-Drash syndrome/Frasier Syndrome, DiGeorge Syndrome (TBX1-associated), Cleidocranial dysplasia, or any other non-CNS-related disorder not listed above.

In certain embodiments of any of the foregoing methods of treatment, the subject is human. In certain embodiments, the method further comprises administering DIACOMIT® (stiripentol), EPIODOLEX® (cannabidiol), a ketogenic diet, ONFI® (clobazam), TOPAMAX® (topiramate), or valproic acid to the subject.

In certain embodiments of any of the foregoing methods, the gene is a SCN1A gene, and the premature stop codon in the SCN1A gene is caused by a mutation, or a combination of mutations, selected from c.664C>T, c.1129C>T, c.1492A>T, c.1624C>T, c.1738C>T, c.1837C>T, c.2134C>T, c.2593C>T, c.3637C>T, c.3733C>T, c.3985C>T, c.4573C>T, c.5656C>T, and c.5734C>T. In certain embodiments, the premature stop is caused by a mutation selected from c.1738C>T and c.3985C>T.

In certain embodiments of any of the foregoing methods, the amino acid is selected from serine, leucine, glutamine and arginine, e.g., the amino acid is selected from glutamine and arginine, e.g., the amino acid is arginine. In certain embodiments, the anticodon hybridizes to a codon selected from UAG, UGA, and UAA, e.g., the anticodon hybridizes to a codon selected from UGA, and UAA, e.g., the anticodon hybridizes to UGA.

In certain embodiments of any of the foregoing methods, the tRNA comprises a nucleotide sequence selected from SEQ ID NO: 1, SEQ ID NO: 2, SEQ ID NO: 19, SEQ ID NO: 20, and SEQ ID NO: 21. In certain embodiments, the tRNA comprises a nucleotide sequence selected from SEQ ID NO: 1 and SEQ ID NO: 2. In certain embodiments, the tRNA comprises one or more naturally occurring nucleotide modifications, e.g., selected from 5-methyl uridine, pseudouridine, dihydrouridine, and 1-methyladenosine.

In certain embodiments of any of the foregoing methods, the expression vector comprises a nucleotide sequence selected from SEQ ID NO: 5, SEQ ID NO: 6, SEQ ID NO: 8, SEQ ID NO: 9, SEQ ID NO: 10, SEQ ID NO: 11, SEQ ID NO: 19, SEQ ID NO: 20, and SEQ ID NO: 21. In certain embodiments, the expression vector comprises a nucleotide sequence selected from SEQ ID NO: 5, SEQ ID NO: 6, SEQ ID NO: 8, SEQ ID NO: 9, SEQ ID NO: 10, and SEQ ID NO: 11. In certain embodiments, the expression vector comprises a nucleotide sequence selected from SEQ ID NO: 19, SEQ ID NO: 20, and SEQ ID NO: 21. In certain embodiments, the expression vector is a viral vector, e.g., a DNA virus vector, e.g., an adeno-associated virus (AAV) vector.

In certain embodiments of any of the foregoing methods, the tRNA or expression vector introduced into the cell or administered to the subject is not conjugated to or associated with another moiety, e.g., a carrier particle, e.g., an aminolipid particle. In certain embodiments, the tRNA or expression vector is introduced into the cell or administered to subject in a dosage form lacking a nanoparticle. In certain embodiments, the tRNA or expression vector is introduced into the cell or administered to subject in a dosage form lacking an aminolipid delivery compound.

In another aspect, this disclosure provides a tRNA that includes the nucleotide sequence of SEQ ID NO: 1, SEQ ID NO: 2, SEQ ID NO: 16, SEQ ID NO: 17, or SEQ ID NO: 18. In certain embodiments, the tRNA comprises the nucleotide sequence of SEQ ID NO: 1 or SEQ ID NO: 2, e.g., the tRNA comprises the nucleotide sequence of SEQ ID NO: 2. In certain embodiments, the tRNA comprises the nucleotide sequence of SEQ ID NO: 16, SEQ ID NO: 17, or SEQ ID NO: 18, e.g., the tRNA comprises the nucleotide sequence of SEQ ID NO: 17 or SEQ ID NO: 18. In certain embodiments, the tRNA comprises a naturally occurring nucleotide modification, e.g., the tRNA comprises one or more nucleotide modifications selected from 5-methyl uridine, pseudouridine, dihydrouridine, and 1-methyladenosine.

In another aspect, this disclosure provides a nucleic acid comprising a nucleotide sequence encoding any of the foregoing tRNAs. In another aspect, this disclosure provides an expression vector comprising the foregoing nucleic acid. In certain embodiments, the expression vector comprises a nucleotide sequence selected from SEQ ID NO: 5, SEQ ID NO: 6, SEQ ID NO: 8, SEQ ID NO: 9, SEQ ID NO: 10, SEQ ID NO: 11, SEQ ID NO: 19, SEQ ID NO: 20, and SEQ ID NO: 21. In certain embodiments, the expression vector comprises a nucleotide sequence selected from SEQ ID NO: 5, SEQ ID NO: 6, SEQ ID NO: 8, SEQ ID NO: 9, SEQ ID NO: 10, and SEQ ID NO: 11. In certain embodiments, the expression vector comprises a nucleotide sequence selected from SEQ ID NO: 19, SEQ ID NO: 20, and SEQ ID NO: 21. In certain embodiments, the expression vector is a viral vector, e.g., a DNA virus vector, e.g., an adeno-associated virus (AAV) vector.

In another aspect, this disclosure provides a pharmaceutical composition comprising any of the foregoing tRNAs or any of the foregoing expression vectors and a pharmaceutically acceptable excipient. In certain embodiments, the tRNA or expression vector is not conjugated to, or associated with, another moiety, e.g., a carrier particle, e.g., an aminolipid particle. In certain embodiments, the composition does not include a nanoparticle and/or an aminolipid delivery compound.

These and other aspects and features of the invention are described in the following detailed description and claims.

### DESCRIPTION OF THE DRAWINGS

FIG. 1 is a schematic representation of SCN1A transcripts containing a premature stop codon (PSC) which leads to a truncated protein product, e.g., a protein product in a subject with Dravet syndrome. Stop codons are indicated as shaded circles, and premature stop codons are indicated as unshaded circles. Expression of a suppressor tRNA (an anticodon modified arginine tRNA) allows read-through of the PSC and facilitates expression of the full-length protein.

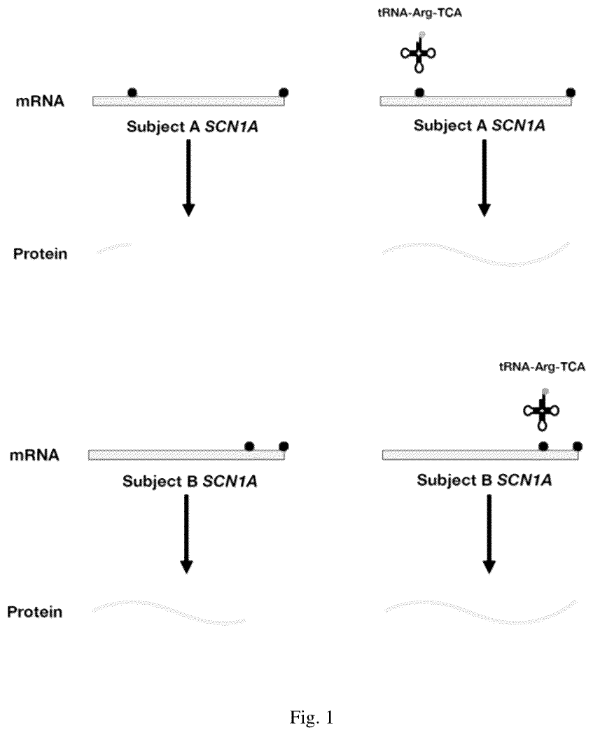

FIG. 2A is a consensus tRNA secondary structure. The numbering of the residues is based on the tRNA numbering system described in Steinberg et al. (1993) NUCLEIC ACIDS RES. 21:3011-15. FIG. 2B is a table showing the modification profile for tRNA sequences from the cytosol of certain eukaryotic organisms. The ratios in the table indicate the frequency of occurrence of listed nucleotide at the numbered position shown in FIG. 2A. The abbreviations for the modified residues are defined in Motorin et al. (2005) “Transfer RNA Modification,” ENCYCLOPEDIA OF LIFE SCIENCES, John Wily & Sons, Inc.

FIG. 3 is a schematic representation of a luciferase reporter construct which contains Renilla luciferase linked to Firefly luciferase via an intervening sequence containing a PSC+/−8 flanking codons.

FIG. 4 shows nucleotide sequences encoding Arg-TCA-1-1 (SEQ ID NO: 1), Arg-TCA-3-1 (SEQ ID NO: 2) and Arg-TCA-6-1 (SEQ ID NO: 3). Mutant TCA anticodons are identified in boxes.

FIGS. 5A-5B are graphs indicating premature stop codon (PSC) read-through in HEK293 cells transfected with the indicated luciferase reporter construct and/or suppressor tRNA. Read-through of the PSC was calculated as the ratio of Firefly luciferase activity to Renilla luciferase activity (Firefly/Renilla). The average readthrough shown on the y-axis is the average readthrough of the PSC containing construct (Firefly/Renilla) divided by the average readthrough of the control CGA containing construct (Firefly/Renilla).

FIG. 6 shows nucleotide sequences encoding Gln-TTA-1-1 (SEQ ID NO: 16), Gln-TTA-2-1 (SEQ ID NO: 17) and Gln-TTA-3-1 (SEQ ID NO: 18). Mutant TTA anticodons are identified in boxes.

### DETAILED DESCRIPTION

This disclosure relates generally to methods and compositions for expressing a gene product encoded by a gene containing a premature stop codon and/or treating a disorder mediated by a premature stop codon.

This disclosure is based, in part, upon the discovery of tRNAs (e.g., suppressor tRNAs), that permit an amino acid to be incorporated into a gene product encoded by a gene in a mammalian cell at a position that would otherwise result in a truncated gene product caused by a premature stop codon (PSC) in the gene. It was further discovered a tRNA that permits an amino acid to be incorporated into a gene product encoded by a gene at a position, which would otherwise result in a truncated gene product caused by a PSC in the gene, can be used to treat a disease mediated by a PSC in a gene in a subject.

Accordingly, in one aspect, this disclosure provides a method of expressing in a mammalian cell a functional gene product encoded by a gene containing a premature stop codon. The method includes introducing into the cell an effective amount of an expression vector capable of expressing a tRNA (e.g., as shown in TABLE 2 below) that (i) comprises an anticodon that hybridizes to the premature stop codon, and (ii) is capable of being aminoacylated with an amino acid, so that the tRNA, when expressed in the cell and aminoacylated with the amino acid, hybridizes to the premature stop codon and permits the amino acid to be incorporated into the gene product at a position that would otherwise result in a truncated gene product caused by the premature stop codon.

In another aspect, this disclosure provides a method of expressing in a mammalian cell a functional gene product encoded by a gene containing a premature stop codon. The method includes introducing into the cell an effective amount of a tRNA (e.g., as shown in TABLE 2 below) that (i) comprises an anticodon that hybridizes to the premature stop codon, and (ii) is capable of being aminoacylated with an amino acid, so that the tRNA, when introduced into the cell and aminoacylated with the amino acid, hybridizes to the premature stop codon and permits the amino acid to be incorporated into the gene product at a position that would otherwise result in a truncated gene product caused by the premature stop codon.

In certain embodiments of any of the foregoing methods, the cell contains less truncated gene product than a cell without the tRNA. For example, in certain embodiments, the cell contains about 5%, about 10%, about 20%, about 30%, about 40%, about 50%, about 60%, about 70%, about 80%, or about 90% of the truncated gene product relative to a cell without the tRNA. In certain embodiments, the cell contains from about 5% to about 80%, about 5% to about 60%, about 5% to about 40%, about 5% to about 20%, about 5% to about 10%, about 10% to about 80%, about 10% to about 60%, about 10% to about 40%, about 10% to about 20%, about 20% to about 80%, about 20% to about 60%, about 20% to about 40%, about 40% to about 80%, about 40% to about 60%, or about 60% to about 80% of the truncated gene product relative to a cell without the tRNA. In certain embodiments, there is no detectable truncated gene product in the cell. Truncated gene product amount or expression may be measured by any method known in the art, for example, Western blot or ELISA.

In certain embodiments, the cell contains a greater amount of functional gene product than a cell without the tRNA. For example, in certain embodiments, the method increases the amount of functional gene product in a cell, tissue, or subject by at least about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, or about 500% relative to a cell, tissue, or subject without the tRNA. In certain embodiments, the method increases the amount of functional gene product in a cell, tissue, or subject, by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject without the tRNA. Functional gene product amount or expression may be measured by any method known in the art, for example, Western blot or ELISA.

In certain embodiments of any of the foregoing methods, the gene is selected from an α-L-iduronidase, ARSB, β-globin, CFTR, CHM, DHCR7, dystrophin, fibrin-1 (FBN1), KIF1A, NAGLU, SCN1A, SMN1, and SMPD1 gene. In certain embodiments, the gene is a SCN1A gene.

In certain embodiments, a premature stop codon in the SCN1A gene is caused by a mutation, or a combination of mutations, selected from c.58G>T, c.575G>A, c.664C>T, c.962C>G, c.1095dupT, c.1129C>T, c.1315C>T, c.1348C>T, c.1366G>T, c.1492A>T, c.1537G>T, c.1624C>T, c.1738C>T, c.1804G>T, c.1837C>T, c.2134C>T, c.2370T>A, c.2495G>A, c.2593C>T, c.2635delC, c.2904C>A, c.3295G>T, c.3311C>A, c.3452C>G, c.3637C>T, c.3656G>A, c.3733C>T, c.3783C>A, c.3829C>T, c.3985C>T, c.4359T>G, c.4547C>A, c.4573C>T, c.4721C>G, c.4954G>T, c.5641G>T, c.5656C>T, and c.5734C>T. In certain embodiments, a premature stop codon in the SCN1A gene is caused by a mutation selected from c.664C>T, c.1129C>T, c.1492A>T, c.1624C>T, c.1738C>T, c.1837C>T, c.2134C>T, c.2593C>T, c.3637C>T, c.3733C>T, c.3985C>T, c.4573C>T, c.5656C>T, and c.5734C>T. In certain embodiments, a premature stop codon in the SCN1A gene is caused by a mutation selected from c.1738C>T and c.3985C>T.

In certain embodiments, wherein the gene is a SCN1A gene, the SCN1A gene product produced with the tRNA is a functional SCN1A gene product. In certain embodiments, the functional SCN1A gene product has greater activity than the truncated SCN1A gene product, e.g., greater voltage-gated sodium channel activity. In certain embodiments, the method increases voltage-gated sodium channel activity in a cell, tissue, or subject by at least about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to a cell, tissue, or subject without the tRNA. In certain embodiments, the method increases voltage-gated sodium channel activity in a cell, tissue, or subject by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject without the tRNA. Voltage-gated sodium channel activity may be measured by any method known in the art, for example, as described in Kalume et al. (2007) J. NEUROSCI. 27(41):11065-74, Yu et al. (2007) NAT. NEUROSCI. 9(9): 1142-9, and Han et al. (2012) NATURE 489(7416): 385-390.

In certain embodiments, the functional SCN1A gene product is the Naᵥ1.1 protein. In certain embodiments, the functional SCN1A gene product is a polypeptide that comprises, consists essentially of, or consists of the amino acid sequence of SEQ ID NO: 4, SEQ ID NO: 14, or SEQ ID NO: 15, or a polypeptide having an amino acid sequence at least 80%, 85%, 86%, 87%, 88%, 89%, 90%, 91%, 92%, 93%, 94%, 95%, 96%, 97%, 98%, or 99% identical to the amino acid sequence of SEQ ID NO: 4, SEQ ID NO: 14, or SEQ ID NO: 15.

In another aspect, this disclosure provides a method of expressing in a cell a functional SCN1A gene product encoded by a SCN1A gene containing a premature stop codon. The method includes introducing into the cell an effective amount of an expression vector capable of expressing a tRNA (e.g., as shown in TABLE 2 below) that (i) comprises an anticodon that hybridizes to the premature stop codon, and (ii) is capable of being aminoacylated with an amino acid, so that the tRNA, when expressed in the cell and aminoacylated with the amino acid, hybridizes to the premature stop codon and permits the amino acid to be incorporated into the SCN1A gene product at a position that would otherwise result in a truncated SCN1A gene product caused by the premature stop codon.

In another aspect, this disclosure provides a method of expressing in a cell a functional SCN1A gene product encoded by a SCN1A gene containing a premature stop codon. The method includes introducing into the cell an effective amount of a tRNA (e.g., as shown in TABLE 2 below) that (i) comprises an anticodon that hybridizes to the premature stop codon, and (ii) is capable of being aminoacylated with an amino acid, so that the tRNA, when introduced into the cell and aminoacylated with the amino acid, hybridizes to the premature stop codon and permits the amino acid to be incorporated into the SCN1A gene product at a position that would otherwise result in a truncated SCN1A gene product caused by the premature stop codon.

In another aspect, this disclosure provides a method of increasing in a cell voltage-gated sodium channel activity encoded by a SCN1A gene containing a premature stop codon. The method includes introducing into the cell an effective amount of an expression vector capable of expressing a tRNA (e.g., as shown in TABLE 2 below) that (i) comprises an anticodon that hybridizes to the premature stop codon, and (ii) is capable of being aminoacylated with an amino acid, so that the tRNA, when expressed in the cell and aminoacylated with the amino acid, hybridizes to the premature stop codon and permits the amino acid to be incorporated into the SCN1A gene product at a position that would otherwise result in a truncated SCN1A gene product caused by the premature stop codon.

In another aspect, this disclosure provides a method of increasing in a cell voltage-gated sodium channel activity encoded by a SCN1A gene containing a premature stop codon. The method includes introducing into the cell an effective amount of a tRNA (e.g., as shown in TABLE 2 below) that (i) comprises an anticodon that hybridizes to the premature stop codon, and (ii) is capable of being aminoacylated with an amino acid, so that the tRNA, when introduced into the cell and aminoacylated with the amino acid, hybridizes to the premature stop codon and permits the amino acid to be incorporated into the SCN1A gene product at a position that would otherwise result in a truncated SCN1A gene product caused by the premature stop codon.

In certain embodiments of any of the foregoing methods, the cell is a human cell. In certain embodiments, the cell is a central nervous system cell, e.g., a neuron.

In another aspect, this disclosure provides a method of treating a premature stop codon-mediated disorder in a subject in need thereof wherein the subject has a gene with a premature stop codon. The method includes administering to the subject an effective amount of an expression vector capable of expressing a tRNA (e.g., as shown in TABLE 2 below) that (i) comprises an anticodon that hybridizes to the premature stop codon, and (ii) is capable of being aminoacylated with an amino acid, thereby to treat the disorder in the subject.

In another aspect, this disclosure provides a method of treating a premature stop codon-mediated disorder in a subject in need thereof, wherein the subject has a gene with a premature stop codon. The method includes administering to the subject an effective amount of a tRNA (e.g., as shown in TABLE 2 below) that (i) comprises an anticodon that hybridizes to the premature stop codon, and (ii) is capable of being aminoacylated with an amino acid, thereby to treat the disorder in the subject.

In certain embodiments, the premature stop codon-mediated disorder is a disorder listed in TABLE 1 below, and the gene with a premature stop codon is a gene listed in the corresponding row of TABLE 1 below.

TABLE 1

| Disorder | Gene |
|---|---|
| β-thalassemia | β-globin |
| Choroideremia | CHM |
| Cystic Fibrosis | CFTR |
| Dravet Syndrome | SCN1A |
| Duchenne Muscular Dystrophy | dystrophin |
| Hurler Syndrome | α-L-iduronidase |
| KIF1A | KIF1A |
| Marfan Syndrome | FBN1 |
| Maroteaux-Lamy Syndrome | ARSB |
| Niemann Pick Disease | SMPD1 |
| Sanfilippo Syndrome | NAGLU |
| Smith-Lemli-Opitz Syndrome | DHCR7 |
| Spinal Muscular Atrophy | SMN1 |

In another aspect, this disclosure provides a method of treating Dravet syndrome in a subject in need thereof wherein the subject has a SCN1A gene with a premature stop codon. The method includes administering to the subject an effective amount of an expression vector capable of expressing a tRNA (e.g., as shown in TABLE 2 below) that (i) comprises an anticodon that hybridizes to the premature stop codon, and (ii) is capable of being aminoacylated with an amino acid, thereby to treat Dravet syndrome in the subject.

In another aspect, this disclosure provides a method of treating Dravet syndrome in a subject in need thereof wherein the subject has a SCN1A gene with a premature stop codon. The method includes administering to the subject an effective amount of a tRNA (e.g., as shown in TABLE 2 below) that (i) comprises an anticodon that hybridizes to the premature stop codon, and (ii) is capable of being aminoacylated with an amino acid, thereby to treat Dravet syndrome in the subject.

In another aspect, this disclosure provides a tRNA comprising the nucleotide sequence of SEQ ID NO: 1, SEQ ID NO: 2, SEQ ID NO: 16, SEQ ID NO: 17, or SEQ ID NO: 18. In certain embodiments, the tRNA comprises the nucleotide sequence of SEQ ID NO: 1 or SEQ ID NO: 2, e.g., the tRNA comprises the nucleotide sequence of SEQ ID NO: 2. In certain embodiments, the tRNA comprises the nucleotide sequence of SEQ ID NO: 16, SEQ ID NO: 17, or SEQ ID NO: 18, e.g., the tRNA comprises the nucleotide sequence of SEQ ID NO: 17 or SEQ ID NO: 18. In another aspect, this disclosure provides a nucleic acid comprising a nucleotide sequence encoding any of the foregoing tRNAs. In another aspect, this disclosure provides an expression vector comprising the foregoing nucleic acid. In another aspect, this disclosure provides a pharmaceutical composition comprising any of the foregoing tRNAs or any of the foregoing expression vectors and a pharmaceutically acceptable excipient.

In certain embodiments of any of the foregoing methods, the tRNA or expression vector introduced into the cell or administered to the subject is not conjugated to or associated with another moiety, e.g., a carrier particle, e.g., an aminolipid particle. In certain embodiments, the tRNA or expression vector is introduced into the cell or administered to subject in a dosage form lacking a nanoparticle. In certain embodiments, the tRNA or expression vector is introduced into the cell or administered to subject in a dosage form lacking an aminolipid delivery compound, e.g., as described in U.S. Patent Publication No. 2017/0354672.

tRNAs and Suppressor tRNAs

During protein synthesis, a transfer RNA (tRNA) delivers an amino acid to a ribosome for incorporation into a growing protein (polypeptide) chain. tRNAs typically are about 70 to 100 nucleotides in length. Active tRNAs contain a 3′ CCA sequence that may be transcribed into the tRNA during its synthesis or may be added later during post-transcriptional processing. During aminoacylation, the amino acid that is attached to a given tRNA molecule is covalently attached to the 2′ or 3′ hydroxyl group of the 3′-terminal ribose to form an aminoacyl-tRNA (aa-tRNA). It is understood that an amino acid can spontaneously migrate from the 2′-hydroxyl group to the 3′-hydroxyl group and vice versa, but it is incorporated into a growing protein chain at the ribosome from the 3′-OH position. A loop at the other end of the folded aa-tRNA molecule contains a sequence of three bases known as the anticodon. When this anticodon sequence hybridizes or base-pairs with a complementary three-base codon sequence in a ribosome-bound messenger RNA (mRNA), the aa-tRNA binds to the ribosome and its amino acid is incorporated into the polypeptide chain being synthesized by the ribosome. Because all tRNAs that base-pair with a specific codon are aminoacylated with a single specific amino acid, the translation of the genetic code is effected by tRNAs. Each of the 61 non-termination codons in an mRNA directs the binding of its cognate aa-tRNA and the addition of a single specific amino acid to the growing polypeptide chain being synthesized by the ribosome.

tRNAs are generally highly conserved and are often functional across species. Accordingly, a tRNA derived from a bacterial tRNA, a non-mammalian eukaryotic tRNA, or a mammalian (e.g., human) tRNA may be useful in the practice of the methods or compositions described herein. Nucleotide sequences encoding naturally occurring human tRNAs are known and generally available to those of skill in the art through sources such as Genbank. See also Sprinzl et al. (2005) NUCLEIC ACIDS RES. 33: D139-40; Buckland et al. (1996) GENOMICS 35(1):164-71; Schimmel et al. (Eds.) (1979) “Transfer-RNA: Structure, Properties, and Recognition,” Cold Spring Harbor Laboratory; Agris (1983) “The Modified Nucleosides of Transfer RNA, II,” Alan R. Liss Inc. tRNAs are generally highly conserved and are often functional across species.

Suppressor tRNAs are modified tRNAs that insert a suitable amino acid at a mutant site, e.g., a PSC, in protein encoding gene. The use of the word in suppressor is based on the fact, that under certain circumstance, the modified tRNA “suppresses” the phenotypic effect of the coding mutation. Suppressor tRNAs typically contain a mutation (modification) in either the anticodon, changing codon specificity, or at some position that alters the aminoacylation identity of the tRNA.

In certain embodiments, a tRNA (e.g., a suppressor tRNA) contains a modified anticodon region, such that the modified anticodon hybridizes with a different codon than the corresponding naturally occurring anticodon. In certain embodiments, the modified anticodon hybridizes with a stop codon, e.g., a PSC, and as a result, the tRNA incorporates an amino acid into a gene product rather than terminating protein synthesis. In certain embodiments, the modified anticodon hybridizes with a premature stop-codon and, and as a result, the tRNA incorporates an amino acid into a gene product at a position that would otherwise result in a truncated gene product caused by the premature stop codon.

In certain embodiments, a tRNA comprises an anticodon that hybridizes to a codon selected from UAG (i.e., an “amber” stop codon), UGA (i.e., an “opal” stop codon), and UAA (i.e., an “ochre” stop codon). In certain embodiments, the anticodon hybridizes to a codon selected from UGA to UAA. In certain embodiments, the anticodon hybridizes to UGA. In certain embodiments, a tRNA comprises an anticodon that hybridizes to a non-standard termination codon, e.g., a 4-nucleotide codon (See, for example, Moore et al. (2000) J. MOL. BIOL. 298:195, and Hohsaka et al. (1999) J. AM. CHEM. SOC. 121:12194).

In certain embodiments, the tRNA is aminoacylated or is capable of being aminoacylated with any natural amino acid. For example, a tRNA may be capable of being aminoacylated with alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In certain embodiments, the tRNA is capable of being aminoacylated with serine, leucine, glutamine, or arginine. In certain embodiments, the tRNA is capable of being aminoacylated with glutamine or arginine. In certain embodiments, the tRNA is capable of being aminoacylated with arginine.

In certain embodiments, the tRNA (i) comprises an anticodon that hybridizes to a codon as indicated in TABLE 2, and (ii) is aminoacylated or is capable of being aminoacylated with an amino acid as indicated in TABLE 2.

TABLE 2

| | | |
|---|---|---|
| codon: UAG | codon: UGA | codon: UAA |
| amino acid: | amino acid: | amino acid: |
| alanine | alanine | alanine |
| codon: UAG | codon: UGA | codon: UAA |
| amino acid: | amino acid: | amino acid: |
| arginine | arginine | arginine |
| codon: UAG | codon: UGA | codon: UAA |
| amino acid: | amino acid: | amino acid: |
| asparagine | asparagine | asparagine |
| codon: UAG | codon: UGA | codon: UAA |
| amino acid: | amino acid: | amino acid: |
| aspartic acid | aspartic acid | aspartic acid |
| codon: UAG | codon: UGA | codon: UAA |
| amino acid: | amino acid: | amino acid: |
| cysteine | cysteine | cysteine |
| codon: UAG | codon: UGA | codon: UAA |
| amino acid: | amino acid: | amino acid: |
| glutamine | glutamine | glutamine |
| codon: UAG | codon: UGA | codon: UAA |
| amino acid: | amino acid: | amino acid: |
| glutamic acid | glutamic acid | glutamic acid |
| codon: UAG | codon: UGA | codon: UAA |
| amino acid: | amino acid: | amino acid: |
| glycine | glycine | glycine |
| codon: UAG | codon: UGA | codon: UAA |
| amino acid: | amino acid: | amino acid: |
| histidine | histidine | histidine |
| codon: UAG | codon: UGA | codon: UAA |
| amino acid: | amino acid: | amino acid: |
| isoleucine | isoleucine | isoleucine |
| codon: UAG | codon: UGA | codon: UAA |
| amino acid: | amino acid: | amino acid: |
| leucine | leucine | leucine |
| codon: UAG | codon: UGA | codon: UAA |
| amino acid: | amino acid: | amino acid: |
| lysine | lysine | lysine |
| codon: UAG | codon: UGA | codon: UAA |
| amino acid: | amino acid: | amino acid: |
| methionine | methionine | methionine |
| codon: UAG | codon: UGA | codon: UAA |
| amino acid: | amino acid: | amino acid: |
| phenylalanine | phenylalanine | phenylalanine |
| codon: UAG | codon: UGA | codon: UAA |
| amino acid: | amino acid: | amino acid: |
| proline | proline | proline |
| codon: UAG | codon: UGA | codon: UAA |
| amino acid: | amino acid: | amino acid: |
| serine | serine | serine |
| codon: UAG | codon: UGA | codon: UAA |
| amino acid: | amino acid: | amino acid: |
| threonine | threonine | threonine |
| codon: UAG | codon: UGA | codon: UAA |
| amino acid: | amino acid: | amino acid: |
| tryptophan | tryptophan | tryptophan |
| codon: UAG | codon: UGA | codon: UAA |
| amino acid: | amino acid: | amino acid: |
| tyrosine | tyrosine | tyrosine |
| codon: UAG | codon: UGA | codon: UAA |
| amino acid: | amino acid: | amino acid: |
| valine | valine | valine |

In certain embodiments, a tRNA comprises, consists essentially of, or consists of the nucleotide sequence of SEQ ID NO: 1, SEQ ID NO: 2, SEQ ID NO: 16, SEQ ID NO: 17, or SEQ ID NO: 18, or a nucleotide sequence having 80%, 85%, 86%, 87%, 88%, 89%, 90%, 91%, 92%, 93%, 94%, 95%, 96%, 97%, 98%, or 99% sequence identity to the nucleotide sequence of SEQ ID NO: 1, SEQ ID NO: 2, SEQ ID NO: 16, SEQ ID NO: 17, or SEQ ID NO: 18.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul (1993) J. MOL. EVOL. 36, 290-300; Altschul et al., (1997) NUCLEIC ACIDS RES. 25:3389-3402) are tailored for sequence similarity searching. For a discussion of basic issues in searching sequence databases see Altschul et al. (1994) NATURE GENETICS 6:119-129. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

It is contemplated that a tRNA may comprise on or more modifications. Exemplary modified tRNAs include: acylated tRNA; alkylated tRNA; a tRNA containing one or more bases other than adenine, cytosine, guanine, or uracil; a tRNA covalently modified by the attachment of a specific ligand or antigenic, fluorescent, affinity, reactive, spectral, or other probe moiety; a tRNA containing one or more ribose moieties that are methylated or otherwise modified; aa-tRNAs that are aminoacylated with an amino acid other than the 20 natural amino acids, including non-natural amino acids that function as a carrier for reagents, specific ligands, or as an antigentic, fluorescent, reactive, affinity, spectral, or other probe; or any combination of these compositions. Exemplary modified tRNA molecules are described in Soll et al. (1995) “tRNA: Structure, Biosynthesis, and Function,” ASM Press; El Yacoubi et al. (2012) ANNU. REV. GENET. 46:69-95; Grosjean et al. (1998) “Modification and Editing of RNA.” ASM Press; Hendrickson et al. (2004) ANNU. REV. BIOCHEM. 73:147-176, 2004; Ibba et al. (2000) ANNU. REV. BIOCHEM. 69:617-650; Johnson et al. (1995) COLD SPRING HARBOR SYMP. QUANT. BIOL. 60:71-82; Johnson et al. (1982) J. MOL. BIOL. 156:113-140; Crowley et al. (1994) CELL 78:61-71; Beier et al. (2001) NUCLEIC ACIDS RES. 29:4767-4782; Tones et al. (2014) TRENDS MOL. MED. 20:306-314; and Bjork et al. (1987) ANNU. REV. BIOCHEM. 56:263-287.

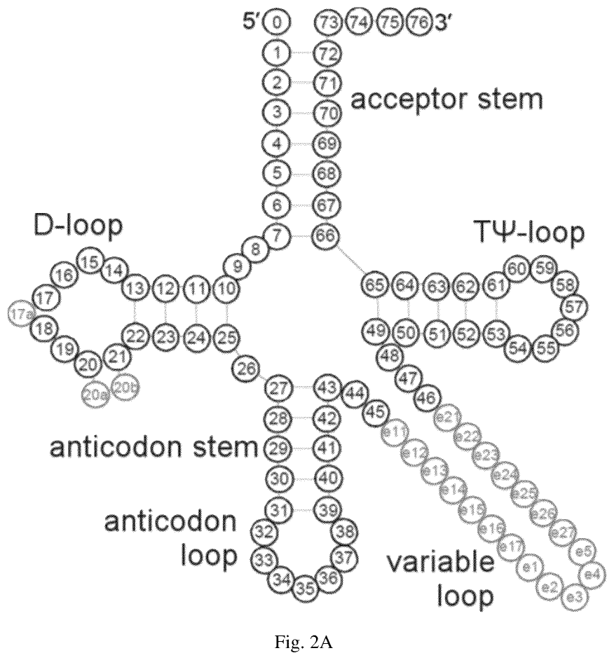

In certain embodiments, a tRNA comprises a naturally occurring nucleotide modification. Naturally occurring tRNAs contain a wide variety of post-transcriptionally modified nucleotides, which are described, for example, in Machnicka et al. (2014) RNA BIOLOGY 11(12): 1619-1629, and include one or more of the residues as shown in FIG. 2B. In certain embodiments, the tRNA comprises one or more of the residues selected from the group consisting of: 2′-O-methylguanosine or G at position 0; pseudouridine or U at position 1; 2′-O-methyladenosine, A, 2′-O-methyluridine, U, 2′-O-methylcytidine, C, 2′-O-methylguanosine, or G at position 4; N2-methylguanosine or G at position 6; N2-methylguanosine or G at position 7; 1-methyladenosine, A, 1-methylguanosine, G, or a modified G at position 9; N2-methylguanosine or G at position 10; N4-acetylcytidine or C at position 12; pseudouridine, U, 2′-O-methylcytidine, or C at position 13; 1-methyladenosine, A, or a modified A at position 14; dihydrouridine (D) or U at position 16; D or U at position 17; 2′-O-methylguanosine or G at position 18; 3-(3-amino-3-carboxypropyl)uridine, D, or U at position 20; 3-(3-amino-3-carboxypropyl)uridine, D, pseudouridine, U, or a modified U at position 20a; D, pseudouridine, or U at position 20b; pseudouridine or U at position 25; pseudouridine, U, N2,N2-dimethylguanosine, N2-methylguanosine, G, or a modified G at position 26; pseudouridine, U, N2,N2-dimethylguanosine, or G at position 27; pseudouridine or U at position 28; pseudouridine or U at position 30; pseudouridine or U at position 31; 2′-O-methylpseudouridine, 2′-O-methyluridine, pseudouridine, U, 2′-O-methylcytidine, 3-methylcytidine, C, or a modified C at position 32; inosine, A, 2-thiouridine, 2′-O-methyluridine, 5-(carboxyhydroxymethyl)uridine methyl ester, 5-carbamoylmethyluridine, 5-carboxymethylaminomethyl-2′-O-methyluridine, 5-methoxycarbonylmethyl-2-thiouridine, 5-methoxycarbonylmethyluridine, pseudouridine, U, a modified U, 2′-O-methylcytidine, 5-formyl-2′-O-methylcytidine, 5-methylcytidine, C, a modified C, queuosine, mannosyl-queuosine, galactosyl-queuosine, 2′-O-methylguanosine, or G at position 34; pseudouridine or U at position 35; pseudouridine, U, or a modified U at position 36; 1-methylinosine, 2-methylthio-N6-threonylcarbamoyladenosine, N6-isopentenyladenosine, N6-methyl-N6-threonylcarbamoyladenosine, N6-threonylcarbamoyladenosine, A, a modified A, 1-methylguanosine, peroxywybutosine, wybutosine, G, or a modified G at position 37; pseudouridine, U, 5-methylcytidine, C, or a modified C at position 38; 1-methylpseudouridine, 2′-O-methylpseudouridine, 2′-O-methyluridine, pseudouridine, U, 2′-O-methylguanosine, or G at position 39; pseudouridine, U, 5-methylcytidine, or C at position 40; 2′-O-methyluridine, U, or a modified U at position 44; pseudouridine or U at position e11; pseudouridine or U at position e12; pseudouridine or U at position e14; 3-methylcytidine or C at position e2; 7-methylguanosine or G at position 46; D, U, or a modified U at position 47; D, U, 5-methylcytidine, C, or a modified C at position 48; A, a modified A, 5-methylcytidine, C, or a modified C at position 49; pseudouridine, U, 5-methylcytidine, or C at position 50; 5,2′-O-dimethyluridine, 5-methyluridine, pseudouridine, or U at position 54; pseudouridine or U at position 55; 1-methyladenosine, A, or a modified A at position 58; 2′-O-ribosyladenosine (phosphate), A, 2′-O-ribosylguanosine (phosphate), G, or a modified G at position 64; pseudouridine or U at position 65; pseudouridine, U, N2-methylguanosine, or G at position 67; pseudouridine or U at position 68; and, pseudouridine, U, 5-methylcytidine, or C at position 72. A, C, G, and U, refer to unmodified adenine, cytosine, guanine, and uracil, respectively. The numbering of the residues is based on the tRNA numbering system described in Steinberg et al., (1993) NUCLEIC ACIDS RES. 21:3011-15.

In certain embodiments, the tRNA comprises one or more nucleotide modifications selected from 5-methyl uridine, pseudouridine, dihydrouridine, and 1-methyladenosine.

Methods of Making tRNAs

It is contemplated the tRNA molecules (e.g., suppressor tRNAs) useful in the methods and compositions described herein can be produced by methods known in the art, including extracellular production by synthetic chemical methods, intracellular production by recombinant DNA methods, or purification from natural sources.

For example, DNA molecules encoding tRNAs can be synthesized chemically or by recombinant DNA methodologies. For example, the sequences of the tRNAs can be synthesized or cloned from libraries by conventional hybridization techniques or polymerase chain reaction (PCR) techniques, using the appropriate synthetic nucleic acid primers. The resulting DNA molecules encoding the tRNAs can be ligated to other appropriate nucleotide sequences, including, for example, expression control sequences to produce conventional gene expression constructs (i.e., expression vectors) encoding the tRNAs. Production of defined gene constructs is within routine skill in the art. Nucleic acids encoding desired tRNAs can be incorporated (ligated) into expression vectors, such as the expression vectors described in the following section, which can be introduced into host cells through conventional transfection or transformation techniques. Examples of host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the tRNAs. Specific expression and purification conditions will vary depending upon the expression system employed.

Alternatively, tRNAs can be chemically synthesized or purified from natural sources by methods known in art. When a tRNA is aminoacylated prior to introduction into the cell or administration to the subject, the tRNA may be aminoacylated with a desired amino acid by any method known in the art, including chemical or enzymatic aminoacylation.

### Expression Vectors

The tRNAs of interest may be expressed in a cell of interest by incorporating a gene encoding a tRNA of interest into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

In certain embodiments, the expression vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Examples of DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the viral vector is a RNA virus vector. Examples of RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain embodiments, the expression vector comprises a regulatory sequence or promoter operably linked to the nucleotide sequence encoding the tRNA. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

tRNA genes can have strong promoters that are active in a variety of cell types. The promoters for eukaryotic tRNA genes typically are present within the structural sequences encoding the tRNA molecule itself. Although there are elements, which regulate transcriptional activity within the 5′ upstream region, the length of an active transcriptional unit may be considerably less than 500 base pairs.

Additional examples of promoters, which may be employed, include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and β-actin promoters). Other viral promoters, which may be employed, include, but are not limited to, adenovirus promoters, TK promoters, and B19 parvovirus promoters. The selection of a promoter will be apparent to those skilled in the art from the teachings contained herein.

In certain embodiments, an expression vector includes a tRNA coding sequence comprising, consisting essentially of, or consisting of the nucleotide sequence of SEQ ID NO: 1, SEQ ID NO: 2, SEQ ID NO: 16, SEQ ID NO: 17, or SEQ ID NO: 18, or a nucleotide sequence having 80%, 85%, 86%, 87%, 88%, 89%, 90%, 91%, 92%, 93%, 94%, 95%, 96%, 97%, 98%, or 99% sequence identity to the nucleotide sequence of SEQ ID NO: 1, SEQ ID NO: 2, SEQ ID NO: 16, SEQ ID NO: 17, or SEQ ID NO: 18.

In certain embodiments, in addition to a tRNA coding sequence, the expression vector comprises a nucleotide sequence corresponding to the genomic DNA sequence flanking a corresponding wild-type tRNA gene. For example, in certain embodiments, an expression vector comprises, consists essentially of, or consists of the nucleotide sequence of SEQ ID NO: 5, SEQ ID NO: 6, SEQ ID NO: 8, SEQ ID NO: 9, SEQ ID NO: 10, SEQ ID NO: 11, SEQ ID NO: 19, SEQ ID NO: 20, or SEQ ID NO: 21, or a nucleotide sequence having 80%, 85%, 86%, 87%, 88%, 89%, 90%, 91%, 92%, 93%, 94%, 95%, 96%, 97%, 98%, or 99% sequence identity to the nucleotide sequence of SEQ ID NO: 5, SEQ ID NO: 6, SEQ ID NO: 8, SEQ ID NO: 9, SEQ ID NO: 10, SEQ ID NO: 11, SEQ ID NO: 19, SEQ ID NO: 20, or SEQ ID NO: 21.

### Adeno-Associated Virus (AAV) Vectors

In certain embodiments, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the methods and compositions described herein can be selected by a skilled person in the art based on the efficiency of delivery, tissue tropism, and immunogenicity. For example, AAV-1, AAV-2, AAV-4, AAV-5, AAV-8, and AAV-9 can be used for delivery to the central nervous system; AAV-1, AAV-8, and AAV-9 can be used for delivery to the heart; AAV-2 can be used for delivery to the kidney; AAV-7, AAV-8, and AAV-9 can be used for delivery to the liver; AAV-4, AAV-5, AAV-6, AAV-9 can be used for delivery to the lung, AAV-8 can be used for delivery to the pancreas, AAV-2, AAV-5, and AAV-8 can be used for delivery to the photoreceptor cells; AAV-1, AAV-2, AAV-4, AAV-5, and AAV-8 can be used for delivery to the retinal pigment epithelium; AAV-1, AAV-6, AAV-7, AAV-8, and AAV-9 can be used for delivery to the skeletal muscle. In certain embodiments, the AAV capsid protein comprises a sequence as disclosed in U.S. Pat. No. 7,198,951, such as, but not limited to, AAV-9 (SEQ ID NOs: 1-3 of U.S. Pat. No. 7,198,951), AAV-2 (SEQ ID NO: 4 of U.S. Pat. No. 7,198,951), AAV-1 (SEQ ID NO: 5 of U.S. Pat. No. 7,198,951), AAV-3 (SEQ ID NO: 6 of U.S. Pat. No. 7,198,951), and AAV-8 (SEQ ID NO: 7 of U.S. Pat. No. 7,198,951). AAV serotypes identified from rhesus monkeys, e.g., rh.8, rh.10, rh.39, rh.43, and rh.74, are also contemplated in the compositions and methods described herein. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous gene of interest. Accordingly, in certain embodiments, the AAV vector comprises a genome comprising an expression cassette for an exogenous gene flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain embodiments, the ITRs are derived from AAV-2. In certain embodiments, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes Ela, E1b55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) CURRENT PROTOCOLS IN MICROBIOLOGY, 14D.1.1-14D.1.21, Production and Discovery of Novel Recombinant Adeno-Associated Viral Vectors). Numerous cell types can be used for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, and 8,163,543, U.S. Patent Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the methods and compositions described herein. Similarly, it is contemplated that any adenoviral type may be used, and a person of skill in the art will be able to identify AAV and adenoviral types that can be used for the production of their desired recombinant AAV vector (rAAV). AAV particles may be purified, for example, by affinity chromatography, iodixonal gradient, or CsCl gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain embodiments, the genome of a self-complementary AAV vector comprises from 5′ to 3′: a 5′ ITR; a first nucleic acid sequence comprising a promoter and/or enhancer operably linked to a coding sequence of a gene of interest; a modified ITR that does not have a functional terminal resolution site; a second nucleic acid sequence complementary or substantially complementary to the first nucleic acid sequence; and a 3′ ITR. AAV vectors containing genomes of all types are can be used in the methods described herein.

Non-limiting examples of AAV vectors include pAAV-MCS (Agilent Technologies), pAAVK-EF1α-MCS (System Bio Catalog #AAV502A-1), pAAVK-EF1α-MC S1-CMV-MC S2 (System Bio Catalog #AAV503A-1), pAAV-ZsGreen1 (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVS1_Puro_PGK1_3×FLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and WO2017201258A1.

In certain embodiments, the expression vector is an AAV vector capable of targeting the nervous system, e.g., the central nervous system, in a subject, e.g., a human subject. Exemplary AAV vectors that can target the nervous system include the AAV9 variants AAV-PHP.B (See, e.g., Deverman et al. (2016) NAT. BIOTECHNOL. 34(2):204-209), AAV-AS (See, e.g., Choudhury et al. (2016) MOL. THER. 24:726-35), and AAV-PHP.eB (See, e.g., Chan et al. (2017) NAT. NEUROSCI. 20:1172-79). Additional exemplary AAV-based strategies for targeting the nervous system are described in Bedrook et al. (2018) ANNU REV NEUROSCI. 41:323-348.

### Lentivirus Vectors

In certain embodiments, the viral vector can be a retroviral vector. Examples of retroviral vectors include moloney murine leukemia virus vectors, spleen necrosis virus vectors, and vectors derived from retroviruses, such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain embodiments, the retroviral vector is a lentiviral vector. Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Often, the structural genes (i.e., gag, pol, and env), are removed from the retroviral backbone using genetic engineering techniques known in the art. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

The genes can be incorporated into the proviral backbone in several general ways. The most straightforward constructions are ones in which the structural genes of the retrovirus are replaced by a single gene that is transcribed under the control of the viral regulatory sequences within the LTR. Retroviral vectors have also been constructed that can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In certain embodiments, the retroviral vector comprises a modified 5′ LTR and/or 3′ LTR. Modifications of the 3′ LTR are often made to improve the safety of lentiviral or retroviral systems by rendering viruses replication-defective. In specific embodiments, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in the methods and compositions described herein.

In certain embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent the 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. VIROLOGY, 69(4):2101-09). The packaging signal may be a minimal packaging signal (also referred to as the psi [Ψ]sequence) needed for encapsidation of the viral genome.

In certain embodiments, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) CELL, 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular embodiments, the retroviral vector backbones comprise one or more FLAP elements upstream or downstream of the heterologous genes of interest in the vectors. For example, in particular embodiments, a transfer plasmid includes a FLAP element. In one embodiment, a vector described herein comprises a FLAP element isolated from HIV-1.

In certain embodiments, the retroviral vector (e.g., lentiviral vector) further comprises an export element. In one embodiment, retroviral vectors comprise one or more export elements. The term “export element” refers to a cis-acting post-transcriptional regulatory element, which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. VIROL. 65: 1053; and Cullen et al., (1991) CELL 58: 423) and the hepatitis B virus post-transcriptional regulatory element (HPRE). Generally, the RNA export element is placed within the 3′ UTR of a gene, and can be inserted as one or multiple copies.

In certain embodiments, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. VIROL., 73:2886); the posttranscriptional regulatory element present in hepatitis B virus (HPRE) (Huang et al., MOL. CELL. BIOL., 5:3864); and the like (Liu et al., (1995), GENES DEV., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain embodiments, vectors described herein lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors described herein lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain embodiments, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector described herein, includes an ideal polyadenylation sequence (e.g., AATAAA, ATTAAA AGTAAA), a bovine growth hormone polyadenylation sequence (BGHpA), a rabbit β-globin polyadenylation sequence (rβgpA), or another suitable heterologous or endogenous polyadenylation sequence known in the art.

In certain embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) PROC. NATL. ACAD. SCI., USA, 99:16433; and Zhan et al., 2001, HUM. GENET., 109:471). In certain embodiments, the retroviral vector comprises an insulator element in one or both LTRs or elsewhere in the region of the vector that integrates into the cellular genome. Examples of insulators for use in the methods and compositions described herein include, but are not limited to, the chicken β-globin insulator (see Chung et al., (1993). CELL 74:505; Chung et al., (1997) PROC. NATL. ACAD. SCI., USA 94:575; and Bell et al., 1999. CELL 98:387). Examples of insulator elements include, but are not limited to, an insulator from a 3-globin locus, such as chicken HS4.

Non-limiting examples of lentiviral vectors include pLVX-EF1alpha-AcGFP1-C1 (Clontech Catalog #631984), pLVX-EF1alpha-IRES-mCherry (Clontech Catalog #631987), pLVX-Puro (Clontech Catalog #632159), pLVX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

### Adenoviral Vectors

In certain embodiments, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serogroup or serotype. Adenoviral serotypes 1 through 51 are available from the American Type Culture Collection (ATCC, Manassas, Virginia). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLoS PATHOG. 5(7):e1000503. A gorilla adenovirus can be used as the source of the adenoviral genome for the adenoviral vector. Gorilla adenoviruses and adenoviral vectors are described in, e.g., PCT Publication Nos. WO2013/052799, WO2013/052811, and WO2013/052832. The adenoviral vector can also comprise a combination of subtypes and thereby be a “chimeric” adenoviral vector.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

In some embodiments, the adenoviral vector is replication-deficient, such that the replication-deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) PROC. NATL. ACAD. SCI. USA 95: 965-976, Chen et al. (1997) PROC. NATL. ACAD. SCI. USA 94: 1645-1650, and Kochanek et al. (1999) HUM. GENE THER. 10(15):2451-9. Examples of replication-deficient adenoviral vectors are disclosed in U.S. Pat. Nos. 5,837,511, 5,851,806, 5,994,106, 6,127,175, 6,482,616, and 7,195,896, and PCT Publication Nos. WO1994/028152, WO1995/002697, WO1995/016772, WO1995/034671, WO1996/022378, WO1997/012986, WO1997/021826, and WO2003/022311.

The replication-deficient adenoviral vector can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. GEN. VIROL. 36: 59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. VIROL. 71: 9206-9213). Other complementing cell lines to produce the replication-deficient adenoviral vector described herein include complementing cells that have been generated to propagate adenoviral vectors encoding transgenes whose expression inhibits viral growth in host cells (see, e.g., U.S. Patent Publication No. 2008/0233650). Additional complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

### Viral Vector Production

Methods for producing viral vectors are known in the art. Typically, a virus of interest is produced in a suitable host cell line using conventional techniques including culturing a transfected or infected host cell under suitable conditions so as to allow the production of infectious viral particles. Nucleic acids encoding viral genes and/or tRNAs can be incorporated into plasmids and introduced into host cells through conventional transfection or transformation techniques. Examples of host cells for production of disclosed viruses include human cell lines, such as HeLa, Hela-S3, HEK293, 911, A549, HER96, or PER-C6 cells. Specific production and purification conditions can vary depending upon the virus and the production system employed.

In certain embodiments, producer cells may be directly administered to a subject, however, in other embodiments, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### Pharmaceutical Compositions

For therapeutic use, a tRNA and/or expression vector preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29). In certain embodiments, the composition does not comprise a nanoparticle or an aminolipid delivery compound, e.g., as described in U.S. Patent Publication No. 2017/0354672. In certain embodiments, the tRNA or expression vector introduced into the cell or administered to the subject is not conjugated to or associated with another moiety, e.g., a carrier particle, e.g., an aminolipid particle. As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically, the moieties are attached either by one or more covalent bonds or by a mechanism that involves specific binding. Alternately, a sufficient number of weaker interactions can provide sufficient stability for moieties to remain physically associated.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing a tRNA and/or expression vector disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a tRNA and/or expression vector is administered intrathecally. In certain embodiments, a tRNA and/or expression vector is administered by injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

In general, any method of delivering a nucleic acid molecule can be adapted for use with a tRNA (see e.g., Akhtar et al. (1992) TRENDS CELL. BIOL. 2(5):139-144 and PCT Publication No. WO94/02595). The tRNA can be modified or alternatively delivered using a drug delivery system to prevent the rapid degradation of the tRNA by endo- and exo-nucleases in vivo. tRNA molecules can be modified by chemical conjugation to lipophilic groups such as cholesterol to enhance cellular uptake and prevent degradation. tRNA molecules can also be conjugated to or otherwise associated with an aptamer. A tRNA can also be delivered using drug delivery systems, such as a nanoparticle, a dendrimer, a polymer, liposomes, or a cationic delivery system. Positively charged cationic delivery systems facilitate binding of a tRNA molecule (negatively charged) and also enhance interactions at the negatively charged cell membrane to permit efficient uptake of a tRNA by the cell. Cationic lipids, dendrimers, or polymers can either be bound to the RNA, e.g., tRNA, or induced to form a vesicle or micelle (see e.g., Kim et al. (2008) JOURNAL OF CONTROLLED RELEASE 129(2):107-116) that encases the RNA. Methods for making and administering cationic-RNA complexes are well within the abilities of one skilled in the art (see, e.g., Sorensen et al. (2003) J. MOL. BIOL 327:761-766; Verma et al. (2003) CLIN. CANCER RES. 9:1291-1300; Arnold et al. (2007) J. HYPERTENS. 25:197-205). Some non-limiting examples of drug delivery systems useful for systemic delivery of RNAs, e.g., tRNAs include DOTAP (Sorensen et al. (2003) supra; Verma et al. (2003), supra), Oligofectamine, solid nucleic acid lipid particles (Zimmermann et al. (2006) NATURE 441:111-114), cardiolipin (Chien et al. (2005) CANCER GENE THER. 12:321-328; Pal et al. (2005) INT J. ONCOL. 26:1087-1091), polyethyleneimine (Bonnet et al. (2008) PHARM. RES. 25(12):2972-82; Aigner (2006) J. BIOMED. BIOTECHOL. 71659), Arg-Gly-Asp (RGD) peptides (Liu (2006) MOL. PHARM. 3:472-487), and polyamidoamines (Tomalia et al. (2007) BIOCHEM. SOC. TRANS. 35:61-67; Yoo et al. (1999) PHARM. RES. 16:1799-1804). In certain embodiments, a tRNA forms a complex with cyclodextrin for systemic administration. Methods for administration and pharmaceutical compositions of RNAs and cyclodextrins can be found in U.S. Pat. No. 7,427,605.

Pharmaceutical formulations can be sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In some embodiments, the pharmaceutical composition is administered subcutaneously and in other embodiments intravenously. Preparations for parenteral administration include sterile aqueous or non-aqueous solutions, suspensions, and emulsions.

Generally, a therapeutically effective amount of active component, for example, a tRNA and/or expression vector, is in the range of 0.1 mg/kg to 100 mg/kg, e.g., 1 mg/kg to 100 mg/kg, 1 mg/kg to 10 mg/kg. In certain embodiments, a therapeutically effective amount of a viral expression vector is in the range of 10² to 10¹⁵ plaque forming units (pfus), e.g., 10² to 10¹⁰, 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life, and the disease being treated. Examples of dosing frequencies are once per day, once per week and once every two weeks. In some embodiments, a route of administration is parenteral, e.g., intravenous infusion.

### Therapeutic Uses

The compositions and methods disclosed herein can be used to treat a premature stop codon (PSC)-mediated disorder in a subject. As used herein, the term “PSC-mediated disorder” refers to a disorder that is mediated, enhanced or otherwise facilitated by or associated with a PSC in a gene. Examples of PSC-mediated disorders include β-thalassemia, Choroideremia (CHM), Cystic Fibrosis, Dravet Syndrome, Duchenne Muscular Dystrophy, Hurler Syndrome, KIF1A, a Lysosomal Storage Disease (e.g., Maroteaux-Lamy Syndrome, Niemann Pick Disease, and Sanfilippo Syndrome), Marfan Syndrome, Smith-Lemli-Opitz Syndrome, and Spinal Muscular Atrophy.

In some embodiments, the PSC-mediated disorder is selected from epilepsy disorders, epileptic encephalopathies, Dravet Syndrome, Lennox-Gastaut Syndrome, Kleefstra Syndrome, Duchenne Muscular Dystrophy; KCNQ2 Encephalopathy, SYNGAP1 Encephalopathy, Parkinson's with GBA, CDKL5, SLC6A1, BRMUTD, Sotos Syndrome, GLUT1 Deficiency Syndrome and any other PSC-mediated disorder associated with a central nervous system (CNS)-related disorder. In some embodiments, the PSC-mediated disorder is selected from epilepsy disorder or epileptic encephalopathies, including Dravet Syndrome and Lennox-Gastaut Syndrome.

In still other embodiments of any of the foregoing methods of treatment, the PSC-mediated disorder is selected 5q-syndrome, Adams-Oliver syndrome 1, Alagille syndrome 1, Autoimmune lymphoproliferative syndrome type 1A, Carney complex type I, CHARGE syndrome, Coffin-Siris Syndrome, Duane Syndrome, Cystic Fibrosis, Marfan Syndrome, Ehlers-Danlos Syndrome, Feingold Syndrome 1, Denys-Drash syndrome/Frasier Syndrome, DiGeorge Syndrome (TBX1-associated), Cleidocranial dysplasia, or any other non-CNS-related disorder not listed above.

In some embodiments, a method of treating a PSC-mediated disorder in a subject in need thereof includes administering to the subject an effective amount of a tRNA and/or expression vector, e.g., a tRNA and/or expression vector disclosed herein, either alone or in a combination with another therapeutic agent to treat the PSC-mediated disorder in the subject.

In certain embodiments, wherein the PSC-mediated disorder is Dravet syndrome, the method reduces seizure frequency, seizure severity, and/or cognitive impairment in the subject. For example, in certain embodiments, the method reduces seizure frequency in the subject by at least 10%, 20%, 30%, 40%, 50%, 60%, 70%, 80%, 90% or 100% over the period of, e.g., a day, a week, or a month. In certain embodiments, the method reduces seizure frequency by 50% over the period of, e.g., a day, a week, or a month.

The term “effective amount” as used herein refers to the amount of an active agent (e.g., tRNA or expression vector described herein) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein is administered in combination with one or more additional therapies, e.g., DIACOMIT® (stiripentol), EPIODOLEX® (cannabidiol), a ketogenic diet, ONFI® (clobazam), TOPAMAX® (topiramate), or valproic acid. For example, during the treatment of Dravet Syndrome, a method or composition described herein is administered in combination with one or more additional therapies, e.g., DIACOMIT® (stiripentol), EPIODOLEX® (cannabidiol), a ketogenic diet, ONFI® (clobazam), TOPAMAX® (topiramate), or valproic acid.

Throughout the description, where compositions are described as having, including, or comprising specific components, or where processes and methods are described as having, including, or comprising specific steps, it is contemplated that, additionally, there are compositions that consist essentially of, or consist of, the recited components, and that there are processes and methods that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition and/or a method described herein can be combined in a variety of ways without departing from the spirit and scope of the compositions and/or methods described herein, whether explicit or implicit herein. For example, where reference is made to a particular compound, that compound can be used in various embodiments of compositions and/or in methods described herein, unless otherwise understood from the context. In other words, within this application, embodiments have been described and depicted in a way that enables a clear and concise application to be written and drawn, but it is intended and will be appreciated that embodiments may be variously combined or separated without parting from the present teachings. For example, it will be appreciated that all features described and depicted herein can be applicable to all aspects of the composition(s) and method(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This examples describes arginine aminoacylated suppressor tRNAs that facilitate read-through of a premature stop codon (PSC) in a SCN1A transcript.

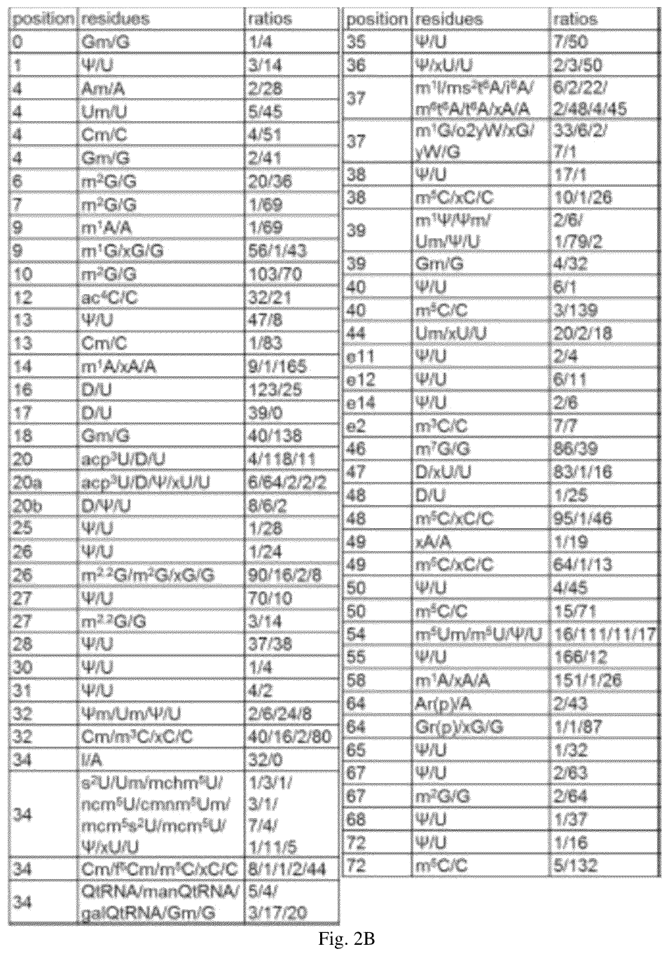

Luciferase reporter constructs were synthesized based on SCN1A transcripts of two Dravet syndrome subjects containing a PSC (subject A and subject B). The reporter construct contained a nucleotide sequence encoding Renilla luciferase and a nucleotide sequence encoding Firefly luciferase linked by an intervening sequence. The intervening sequence contained the PSC and eight flanking codons from the SCN1A transcript of subject A (SEQ ID NO: 12) and subject B (SEQ ID NO: 13) (FIG. 3). The reporter constructs are hereafter referred to as the subject A and subject B reporter constructs. For each of the subject A and subject B reporter constructs, a control reported construct was synthesized that was the same except for a CGA (arginine) codon that was inserted in place of the PSC.

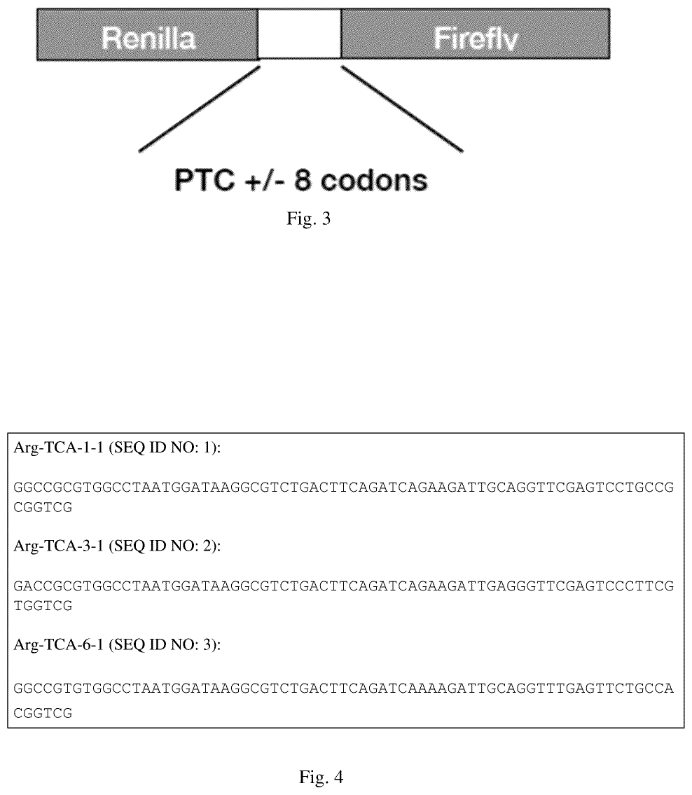

Suppressor tRNAs were derived from tRNA-Arginine genes (tRNA-Arg-TCG-1-1, tRNA-Arg-TCG-3-1, and tRNA-Arg-TCG-6-1). In each RNA, the TCG anticodon was mutated to TCA, producing tRNAs referred to as Arg-TCA-1-1 (SEQ ID NO: 1), Arg-TCA-3-1 (SEQ ID NO: 2) and Arg-TCA-6-1 (SEQ ID NO: 3). Arg-TCA-1-1 (SEQ ID NO: 1), Arg-TCA-3-1 (SEQ ID NO: 2) and Arg-TCA-6-1 (SEQ ID NO: 3) are depicted in FIG. 4, with the mutant TCA anticodon indicated.

Nucleotide sequences encoding the Arg-TCA-1-1 tRNA along with 200 base pairs (bp) of flanking genomic DNA sequence from the corresponding wild-type tRNA gene (SEQ ID NO: 5), the Arg-TCA-3-1 tRNA along with 200 bp of flanking genomic DNA sequence from the corresponding wild-type tRNA gene (SEQ ID NO: 6), and the Arg-TCA-6-1 tRNA along with 200 bp of flanking genomic DNA sequence from the corresponding wild-type tRNA gene (SEQ ID NO: 7) were cloned into pTRE-Tight (Clontech) using XhoI and PciI restriction enzyme sites, which resulted in removal of the Ptight, MCS, and SV40 poly A sequences.

HEK293 cells grown in DMEM with 10% FBS to 30-40% confluence were co-transfected with reporter constructs and the tRNAs using Effectene (Qiagen). Twenty-four hours later, cells were harvested and Firefly and Renilla luciferase activities were determined using the Dual-Luciferase® Reporter Assay System (Promega).

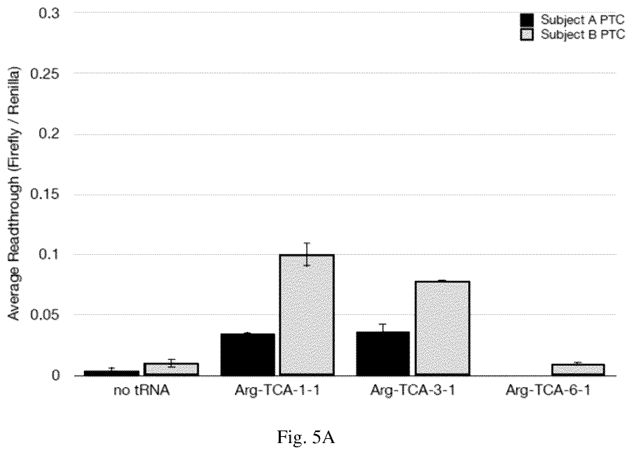

As shown in FIG. 5A, expression of the synthetic Arg-TCA-1-1 and Arg-TCA-3-1 suppressor tRNAs facilitated read-through of both the subject A and subject B reporter constructs.

In addition, Arg-TCG-1-1 was tested with varying flanking or regulatory sequences. A nucleotide sequence encoding the Arg-TCA-1-1 tRNAs along with (i) 46 bp of flanking genomic DNA sequence from the corresponding wild-type tRNA gene (“minimal”; SEQ ID NO: 8), (ii) an upstream U6 promoter plus 46 bp of flanking genomic DNA sequence from the corresponding wild-type tRNA gene (“U6P+minimal”; SEQ ID NO: 10) or (iii) 200 bp of flanking genomic DNA sequence from the corresponding wild-type tRNA gene (“+/−200”; SEQ ID NO: 5) was cloned into pTRE-Tight as described above.

HEK-293 cells were co-transfected with the tRNA expressing constructs and the subject B luciferase reporter construct. Cell transfection, luminescence detection, and read-through calculation were performed as described above. As shown in FIG. 5B, expression of each of the minimal, U6P+minimal, and +/−200 bp tRNA-Arg-TCA-1-1 constructs facilitated read-through of the subject B reporter constructs.

Together, these results demonstrate that the described suppressor tRNAs can facilitate expression of SCN1A transcripts containing premature stop codons associated with Dravet syndrome.

### Example 2

This example describes glutamine aminoacylated suppressor tRNAs that facilitate read-through of a premature stop codon (PSC) in a transcript.

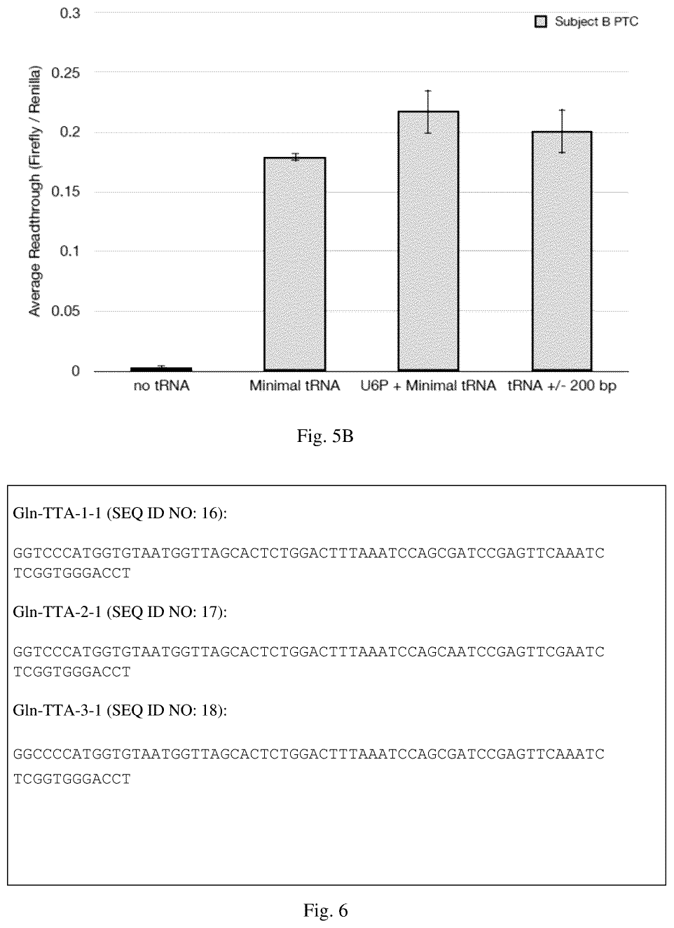

Suppressor tRNAs are derived from tRNA-Glutamine genes (tRNA-Gln-TTG-1-1, tRNA-Gln-TTG-2-1, and tRNA-Gln-TTG-3-1). In each RNA, the TTG anticodon is mutated to TTA, producing tRNAs referred to as Gln-TTA-1-1 (SEQ ID NO: 16), Gln-TTA-2-1 (SEQ ID NO: 17) and Gln-TTA-3-1 (SEQ ID NO: 18). Gln-TTA-1-1 (SEQ ID NO: 16), Gln-TTA-2-1 (SEQ ID NO: 17) and Gln-TTA-3-1 (SEQ ID NO: 18) are depicted in FIG. 6, with the mutant TTA anticodon indicated.

Nucleotide sequences encoding the Gln-TTA-1-1 tRNA along with 200 base pairs (bp) of flanking genomic DNA sequence from the corresponding wild-type tRNA gene (SEQ ID NO: 19), the Gln-TTA-2-1 tRNA along with 200 bp of flanking genomic DNA sequence from the corresponding wild-type tRNA gene (SEQ ID NO: 20), and the Gln-TTA-3-1 tRNA along with 200 bp of flanking genomic DNA sequence from the corresponding wild-type tRNA gene (SEQ ID NO: 21) are cloned into pTRE-Tight (Clontech) using XhoI and PciI restriction enzyme sites, which results in removal of the Ptight, MCS, and SV40 poly A sequences.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-69. (canceled)

70. A method of treating a premature stop codon-mediated disorder in a subject in need thereof, the method comprising:
administering to the subject an effective amount of an expression vector capable of expression of a tRNA, wherein the tRNA (i) comprises an anticodon that hybridizes to the premature stop codon, and (ii) is capable of being aminoacylated with an amino acid, thereby to treat the genetic disease or disorder in the subject.

71. The method of claim 70, wherein the subject has an SCN1A gene with a premature stop codon mutation.

72. The method of claim 71, the SCN1A gene premature stop codon mutation selected from c.664C>T, c.1129C>T, c.1492A>T, c.1624C>T, c.1738C>T, c.1837C>T, c.2134C>T, c.2593C>T, c.3637C>T, c.3733C>T, c.3985C>T, c.4573C>T, c.5656C>T, and c.5734C>T.

73. The method of claim 70, wherein the expression vector comprises a nucleotide sequence selected from SEQ ID NO: 1, SEQ ID NO: 2, SEQ ID NO: 3, SEQ ID NO: 16, SEQ ID NO: 17, and SEQ ID NO: 18.

74. The method of claim 71, wherein the SCN1A gene premature stop codon mutation is a mutation selected from c.17380>T and c.3985C>T.

75. The method of claim 70, wherein the amino acid is selected from serine, leucine, glutamine, and arginine.

76. The method of claim 70, wherein the expression vector comprises the nucleotide sequence selected from SEQ ID NO: 1, SEQ ID NO: 2, and SEQ ID NO: 3.

77. The method of claim 70, wherein the expression vector comprises a nucleotide sequence selected from SEQ ID NO: 5, SEQ ID NO: 6, SEQ ID NO: 8, SEQ ID NO: 9, SEQ ID NO: 10, SEQ ID NO: 11, SEQ ID NO: 19, SEQ ID NO: 20, and SEQ ID NO: 21.

78. The method of claim 70, wherein the expression vector comprises a nucleotide sequence selected from SEQ ID NO: 5, SEQ ID NO: 8, and SEQ ID NO: 10.

79. The method of claim 70, wherein the premature stop codon-mediated disorder is a premature stop codon-mediated disorder associated with a central nervous system (CNS)-related disorder.

80. The method of claim 79, wherein the CNS-related disorder is an epilepsy disorder or epileptic encephalopathy selected from the group consisting of Dravet Syndrome, Lennox-Gastaut Syndrome, Kleefstra Syndrome, Duchenne Muscular Dystrophy, KCNQ2 Encephalopathy, SYNGAP1 Encephalopathy, Parkinson's with GBA, CDKL5, SLC6A1, BRMUTD, Sotos Syndrome, and GLUT1 Deficiency Syndrome.

81. The method of claim 70, wherein the expression vector is provided in a pharmaceutical composition with a pharmaceutically acceptable excipient.
